(19) United States
(12) Patent Application Publication
(10) Pub. No.: US 2024/0395369 A1
(43) Pub. Date: Nov. 28, 2024

(54) SYSTEM AND METHOD FOR AUTOMATING MEDICAL HISTORY DOCUMENTATION USING ARTIFICIAL INTELLIGENCE

(71) Applicants: Senthilkumar Mariappan, Columbus, GA (US); Karthick Sankaranarayanan, Alpharetta, GA (US); Arunbabu Sankaranarayanan, Dayton, OH (US)

(72) Inventors: Senthilkumar Mariappan, Columbus, GA (US); Karthick Sankaranarayanan, Alpharetta, GA (US); Arunbabu Sankaranarayanan, Dayton, OH (US)

(21) Appl. No.: 18/671,658

(22) Filed: May 22, 2024

Related U.S. Application Data
(60) Provisional application No. 63/468,046, filed on May 22, 2023.

(51) Int. Cl. G16H 10/20; G16H 10/60

(52) U.S. Cl. CPC G16H 10/20; G16H 10/60

(57) ABSTRACT

An automated medical documentation system allows a user to access the data center of a medical care enterprise to enter basic personal and medical information that is kept in a record. The information is provided verbally, using natural language processing, and information provided by the patient is identified for further questioning. Questions can follow a diagnosis tree and establish the basic facts of the patient's illness or ailment. The system generates a visual identifier that can be scanned and used to automatically access the patient's record once the patient arrives at the medical facility. The same identifier can be used for automated processing of test samples.

### CROSS-REFERENCE TO RELATED APPLICATION

This application claims priority to U.S. Provisional Patent Application No. 63/468,046, filed May 22, 2023, the entirety of which is hereby incorporated by reference.

### FIELD OF THE INVENTION

Embodiments of the invention relate to systems and methods for the automated entry of documentation of medical history, and to providing the medical history to healthcare providers.

### BACKGROUND OF THE INVENTION

Medical history-taking can be time-consuming, leading to healthcare providers spending more time on documentation and hence physician burnout. Additionally, redundant history-taking by various providers at different levels of care can lead to inefficiencies and wasted resources. The documentation contributes to the lengthy wait times in Emergency Rooms, Medical Offices and sometimes delays in the patient's care.

A typical patient experience exemplified in the following process. A patient at home, or any location outside the hospital, experiences severe pain or discomfort significant enough to cause concern. This prompts the decision to seek emergency medical care. After traveling for a variable duration, the patient arrives at the emergency room (ER). Upon entering the ER, the patient undergoes a registration process where personal details such as name, ID verification, and insurance information are collected, typically taking 5-10 minutes. Following registration, the patient may experience a wait before seeing a triage nurse. During the triage, the nurse records a brief summary of the patient's health concerns along with vital signs including blood pressure, heart rate, and oxygen saturation. This initial assessment also takes about 5-10 minutes, but the patient may continue to wait until an ER bed becomes available. The average wait time in U.S. ERs is 90 minutes, though it can extend to several hours depending on the facility's capacity and crowd. Once assigned to an ER bed, the patient encounters an ER provider who initiates the medical history-taking process. This crucial stage involves detailed inquiries about the patient's primary complaint, associated symptoms, and comprehensive health background-including past medical, family, and social histories. These inquiries are structured to refine the differential diagnosis, probing deeply to confirm or eliminate potential conditions. The provider then decides on immediate investigations and treatments aimed at symptom management and stabilization. Depending on the patient's response and the need for further care, they may either be discharged or recommended for hospital admission. If admission is deemed necessary, the ER provider alerts the admitting physician, typically a hospitalist or internal medicine specialist. While waiting for the hospitalist, the ER provider compiles a detailed document summarizing the history of the present illness. This documentation process requires an additional 5-10 minutes. Upon the hospitalist's arrival, they engage in a more thorough exploration of the patient's medical history and current medications. This stage often demands 10-15 minutes for history taking and an equal duration for documentation, depending on the case's complexity. Should the patient's condition warrant specialist consultation, they advance to the next stage, which might involve multiple sub-stages, depending on the number of specialists required (e.g., cardiologists, nephrologists). Each specialist typically spends 10-15 minutes on history taking and an equal amount on documentation. This repetitive process can be frustrating and exhausting for you as a patient, especially when you are in pain and need to recount your symptoms multiple times. From a provider's perspective, this redundancy consumes valuable time that could otherwise be spent treating patients. On average, each provider spends between 15-30 minutes on history taking per patient, per stage of care. Multiply this by the number of patients seen daily, and the cumulative time spent on just collecting and documenting history is substantial.

The inefficiencies of the above typical process can have substantial negative effects on patient satisfaction, provider burnout, and operational costs. Repeating the same information multiple times can aggravate patients, reducing their overall satisfaction with the care process, especially if their pain and symptoms are severe. The monotonous task of documenting extensive medical histories under time pressure contributes significantly to healthcare provider burnout. This not only affects their well-being but can also impact the quality of care delivered. The redundancy and inefficiencies in the history-taking process are costly, not just in terms of time but also in the broader economic impact on healthcare systems.

Therefore, a need exists to overcome the problems with the prior art as discussed above.

### SUMMARY OF THE INVENTION

This summary is provided to introduce a variety of concepts in a simplified form that is disclosed further in the detailed description of the embodiments. This summary is not intended for determining or limiting the scope of the claimed subject matter.

The embodiments provided herein relate broadly to a system for automatically recording medical history, wherein at least one user computing device is in operable connection with a user network. An application server is in operable communication with the user network to host an application system for automatically recording medical history, the application system having a user interface module for providing access to the application program through the user computing device.

The embodiments use Natural Language Process (NLP) techniques using Large Language Models (LLM) to understand and process user inputs, identify intents and entities, and generate appropriate responses. It incorporates a comprehensive knowledge base of medical information, including symptoms, diseases, and diagnostic criteria, to generate differential diagnoses and History of Present Illness (HPI) documentation. In this way, time spent on medical history is taken at various provider levels, reducing Emergency Room wait times. The generated documentation can also be used for Medical Offices and to reduce Patient wait times.

The problems with existing history-taking systems include the manual process of taking and documenting the patient's history, which is time-consuming and can sometimes be prone to errors and omissions.

Existing history-taking systems don't work well because they rely heavily on manual data entry and may be subject to human error and bias. They also may not be easily accessible or available to all medical providers, leading to redundant history-taking.

In accordance with some embodiments of the inventive disclosure, there is provided a method and system for automating medical documentation that includes receiving, at a data center, speech provided by a patient computing device located remotely from the data center via a wide area network. The embodiments further include processing said speech to identify keywords relating to at least one medical condition and generating at least one follow up question in a natural language prompt that is transmitted to the patient computing device and receiving follow up information via speech from the patient computing device. The embodiments further include generating a visual identifier that is then associated with a record created to include the relevant information obtained from the speech and transmitting the visual identifier to the patient computing device. The embodiments further include receiving, at the data center, from a check-in kiosk at a medical facility associated with the data center, information from the visual identifier. The embodiment also include the data center, in response to receiving the information from the visual identifier, accessing the record and pushing information from the record to at least one computing device associated with medical care provider at the medical facility, wherein pushing the information prevents the need to ask a patient associated with the patient computing device questions that have been answered.

Although the invention is illustrated and described herein as embodied in a system and method for automating medical history documentation, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

“In the description of the embodiments of the present invention, unless otherwise specified, azimuth or positional relationships indicated by terms such as “up”, “down”, “left”, “right”, “inside”, “outside”, “front”, “back”, “head”, “tail” and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first”, “second”, “third” and so on are only used for descriptive purposes, and cannot be construed as indicating or implying relative importance.

In the description of the embodiments of the present invention, it should be noted that, unless otherwise clearly defined and limited, terms such as “installed”, “coupled”, “connected” should be broadly interpreted, for example, it may be fixedly connected, or may be detachably connected, or integrally connected; it may be mechanically connected, or may be electrically connected; it may be directly connected, or may be indirectly connected via an intermediate medium. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system. Those skilled in the art can understand the specific meanings of the above-mentioned terms in the embodiments of the present invention according to the specific circumstances.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 illustrates a system architecture diagram, according to some embodiments.

FIG. 2 shows a block diagram of an example of a medical record system, in accordance with some embodiments.

FIG. 3 shows a chart of an initial process for receiving a patient at a medical facility, in accordance with some embodiments.

FIG. 4 shows a flow chart diagram of a method for receiving a patient at a medical facility, in accordance with some embodiments.

FIG. 5 shows a flow chart diagram of a method for processing a patient in a medical facility, in accordance with some embodiments.

FIG. 6 shows a diagram illustrating a process for initiating, tracking, and processing laboratory samples in relation to diagnosing a patient, in accordance with some embodiments.

FIG. 7 shows a network diagram of a system for initiating and using medical records, in accordance with some embodiments.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relates to systems and methods for automating medical record taking tasks by extracting information from users based on their symptoms. The embodiments use Natural Language Process (NLP) techniques using Large Language Model (LLM) to understand and process user inputs, identify intents and entities, and generate appropriate responses. It incorporates a comprehensive knowledge base of medical information, including symptoms, diseases, and diagnostic criteria, to generate differential diagnoses and History of Present Illness (HPI) documentation. In this way, time spent on medical history is taken at various provider levels, reducing Emergency Room wait times. The generated documentation can also be used for Medical Offices and to reduce Patient wait times.

During use, the user first enters the platform and selects the symptoms which are applicable. The user then selects their primary problem, and the system begins the Chief Compliant task which asks questions about character, location, and duration. The software asks questions about individual symptomatology. The system then compiles the information in a way that is readily understandable by a medical professional. Next, the user provides past medical history through individual interactive icons for medical diseases. The user then provides surgical history through individual descriptions in anatomy, using interactive touchable options for quick input and the system uses text summarization to compile the entire HPI in a documentation format at EMR standards. The HPI is securely stored in the database. The user selects “Finish” to generate a comprehensive HPI document. The HPI document is automatically integrated with the EMR system via APIs or QR scanning or can be manually uploaded by an administrator through the hospital/office admin page.

The embodiments use Natural Language Processing (NLP) methods using Large Language Model (LLM) to allow automated history taking and documentation, making it better than existing history-taking solutions. The system also reduces the burden on healthcare providers and improves the accuracy and completeness of the History of Present Illness (HPI) documentation to providers at all levels.

The system improves on existing history-taking systems by using natural language processing (NLP) using a Large Language Model (LLM) to streamline the history-taking and documentation process, reducing the risk of errors and omissions, and improving overall efficiency. The system also easily accessible and available to medical providers at any time, avoiding the need for repetitive history-taking.

The user interface allows healthcare providers to input the patient's information and access the generated History of Present Illness (HPI) and other documentation. The Natural Language Process (NLP) framework includes algorithms for text classification, sentiment analysis, entity recognition, and language translation. The system stores patient history and History of Present Illness (HPI) documentation securely in a database. The system provides Application Programming Interface (API) or QR Scanning allows integration of EHR systems and other third-party tools.

The User Interface is the front-end component of the invention that allows healthcare providers to input patient information and access the generated History of Present Illness (HPI) and other documentation. The Natural Language Process (NLP) Framework using Large Language Model (LLM) includes the algorithms for text classification, sentiment analysis, entity recognition, and language translation, which are used to analyze the patient's history and generate the History of Present Illness (HPI) and other documentation. The system stores patient history and History of Present Illness (HPI) and other documentation securely in a HIPAA compliance database for future use. History of Present Illness (HPI) and other document Integration An application programming interface (API) created using open API standards or QR Scanning allows electronic health record (EHR) systems and other third-party tools to access the data with ease, allowing for seamless information sharing between different healthcare providers and institutions. The different components work together to generate accurate and comprehensive History of Present Illness (HPI) and other documentation that healthcare providers can use to make informed decisions about patient care.

In some embodiments, Natural Language Process (NLP) framework using Large Language Model (LLM) follows a series of logical steps to process the patient's history and generate the History of Present Illness (HPI) and other documentation. The system collects the patient's history either through direct input from the healthcare provider or by integrating with third-party tools that have the patient's medical records. The system pre-processes the input data, which involves techniques like tokenization, sentence segmentation, and stop-word removal. The system identifies important entities like diseases, symptoms, medications, and treatments mentioned in the patient's history. The system classifies the input data into relevant categories like past medical history, family history, social history, etc. The system performs sentiment analysis on the input data to determine the patient's emotional state and provide insight into their mental health. If necessary, the system translates the input data into the required locale language for processing. Using the processed input data, the system generates a comprehensive and accurate History of Present Illness (HPI) and other documentation, ready to be integrated with the electronic medical records system (EMR).

The system first captures a patient's history through a user-friendly interface and structured interview process. Once the interview is complete, the system applies Natural Language Process (NLP) algorithms using health care Large Language Model (LLM), including text classification, sentiment analysis, entity recognition, and locale language translation, to extract and categorize relevant information from the patient's history.

The system then identifies redundant or irrelevant information and removes it to produce a concise and clinically relevant summary of the patient's History of Present Illness (HPI). This summary is then structured into a narrative format that is compliant with the SOAP (Subjective, Objective, Assessment, and Plan) note format, which is commonly used in Electronic Medical Record (EMR) systems. The generated History of Present Illness (HPI) and other documentation is then securely stored in a database, which can be accessed by healthcare providers through the platform's API for integration with Electronic Medical Record (EMR) systems and other third-party tools. By automating the process of generating History of Present Illness (HPI) and other documentation, the application enables healthcare providers to save time and improve accuracy while ensuring compliance with industry standards. The system's user-friendly interface, advanced Natural Language Process (NLP) algorithms using health care Large Language Model (LLM), and secure data storage make it a powerful tool for enhancing the efficiency and effectiveness of healthcare delivery. Develop the user interface: The user interface can be developed using a javascript framework like React or Angular and UX/wireframes can be designed using diagram tools like figma or Adobe. Develop the NLP framework: The Natural Language Process (NLP) framework can be developed using Java/Python libraries like spaCy, NLTK, and TensorFlow. The algorithms for text classification, sentiment analysis, entity recognition, and language translation can be developed using these libraries.

The database for the system can be developed using relational/NoSQL databases like MySQL/AWS DynamoDb or MongoDB. The database schema can be designed based on the requirements of the system and the data can be stored securely using strong encryption mechanism like Google's Tink library. The key can be managed by cloud services like AWS/GCP. The Application Programming Interface (API) s for integration with Electronic Health Record (EHR) systems and other third-party tools can be developed using programming languages like Java or Python and can be designed using frameworks like Spring or Flask. The user interface, NLP framework, data storage, and APIs can be integrated using a micro services architecture. This involves breaking down the system into small, independent services that can communicate with each other through APIs. The system can be tested using automated testing frameworks like JUnit or PyTest. This involves testing the functionality of each component and the integration of the system as a whole. The system can be deployed on a cloud platform like AWS or Google Cloud Platform. This involves configuring the system for scalability, availability, and security. The system will be maintained regularly to ensure its performance and security. This involves monitoring the system, updating the software and hardware components, and fixing any issues that arise.

Necessary elements for the invention include the Front-end user interface, Natural Language Process (NLP) framework, data storage, and Application Programming Interface (API) s. Elements include additional features for the Front-end user interface, such as customization options or additional input fields. The Natural Language Process (NLP) framework could be improved with more sophisticated algorithms or expanding Large Language Model (LLM) support. Additional features include increasing data storage capabilities or integration with other third-party tools.

Once admitted, the patient's daily progress notes, including subjective details and concerns, can be entered by the patient or hospital user (e.g., nursing staff, Patient Care Technician PCT using the EHR system. The entered progress notes are securely stored in the EHR system. They can be reviewed by the physician before or during the morning rounds, providing a comprehensive overview of the patient's condition and concerns even before seeing the patient.

Seamless ER Admission: Enables the patient to enter their HPI using the inventive application program from home anywhere at the patient's convenience, which securely transmits the entered HPI to the designated hospital. Upon arrival at the hospital's Emergency Room (ER), the patient's information, including their HPI, is automatically triaged and assigned to an ER provider based on their arrival time. The assigned ER provider can access the patient's HPI and other relevant information from the EHR system, allowing for a seamless admission process and continuity of care.

Clinical decision support such as health care companies can incorporate evidence-based guidelines and algorithms to assist providers in making clinical decisions. These elements include alerts for potential diagnoses or treatment options, and recommendations for appropriate patient's follow-up care. Health care companies can allow for seamless integration with Telemedicine and Online Health care consulting platforms, enabling providers to conduct virtual visits with patients from within the same system. This can increase accessibility and convenience for patients, also allowing providers to conduct remote assessments, consulting and follow-up care. The Natural Language Process (NLP) framework could be replaced with a different algorithmic approach, or a different database system could be used for data storage. The user interface could be redesigned to have a different layout or incorporate different features. Different Application Programming Interface (API) s could be used for integration with different Electronic Health Record (EHR) systems or third-party tools. The logic of the system could be modified to incorporate additional steps or different algorithms. Different encryption mechanisms could be applied to achieve the same secure systems. Different cloud providers can be chosen to deploy the required components. These changes would allow the invention to perform similar functions. To use the application program to solve the problem of inefficient and error-prone patient history collection and documentation, a healthcare provider would follow these steps: Open the applications user interface on a computer or mobile device and enter the patient's basic information. Ask the patient to provide their medical history, by using either voice assistant or touch screen interface to enter the information on their own. Hospitals and Medical offices can provide kiosks using iPad or any other similar devices with the application for patients who don't have access to mobile devices or to mobile apps. Once the patient's history has been collected, the healthcare provider can integrate, review, edit and save the information as needed. Click the “Generate HPI Documentation” button, which will trigger the Natural Language Process (NLP) framework to analyze the patient's history and generate a comprehensive History of Present Illness (HPI) document. Which can be used by providers at all levels including the consultants. Review the generated History of Present Illness (HPI) document and make any necessary edits or additions. Save the History of Present Illness (HPI) document securely to the database, and optionally export it to the Electronic Health Record (EHR) system via the provided APIs or other third-party tools. Steps mentioned above can be used for Daily Patient Progress note for Subjective details as well.

By following these steps, a healthcare provider can efficiently and accurately collect and document patient history using the application, reducing the risk of errors and saving time compared to traditional manual methods. The proposed invention can be adapted and used in various fields where patient data is collected and analyzed, such as clinical research, insurance underwriting, and population health management. For example, in clinical research, the Natural Language Process (NLP) framework can be used to automatically extract relevant information from clinical trial documents and generate summaries for researchers. In insurance underwriting, the system can be used to assess a patient's health risk by analyzing their medical history and generating a risk assessment report. In population health management, the system can be used to analyze large-scale patient data to identify patterns and trends, leading to improved healthcare outcomes at the population level.

For patients, the application saves time during appointments by streamlining the history taking process and early treatment. Reduces the need for patients to repeat their medical history at every visit. Improves accuracy of medical information, leading to better diagnosis and treatment. Increases patient engagement and involvement in their own care.

For Physicians, the application saves time during appointments and documentation, reducing physician burnout. Increases accuracy of medical information, leading to better diagnosis and treatment. Improves communication between healthcare providers. Provides decision support tools to assist in clinical decision-making.

For Hospitals, the application saves provider time, potentially resulting in cost savings. Decreases the average ER wait time. Improves the quality of care and patient outcomes. Streamlines the billing and reimbursement process.

At a public health level, if prevalently adopted it could decrease overall Emergency Room wait times in the US.

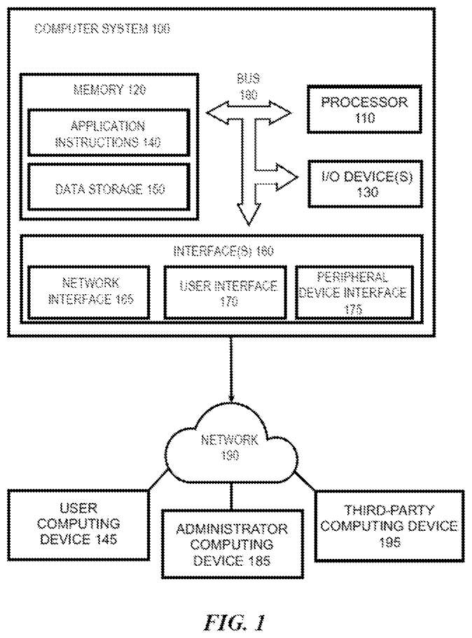

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

As used herein, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random-access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized a user (e.g., a healthcare provider) to interact with the various functionalities of the system including to perform patient rounds, handoff patient rounding responsibility, perform biometric verification tasks, and other associated tasks and functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

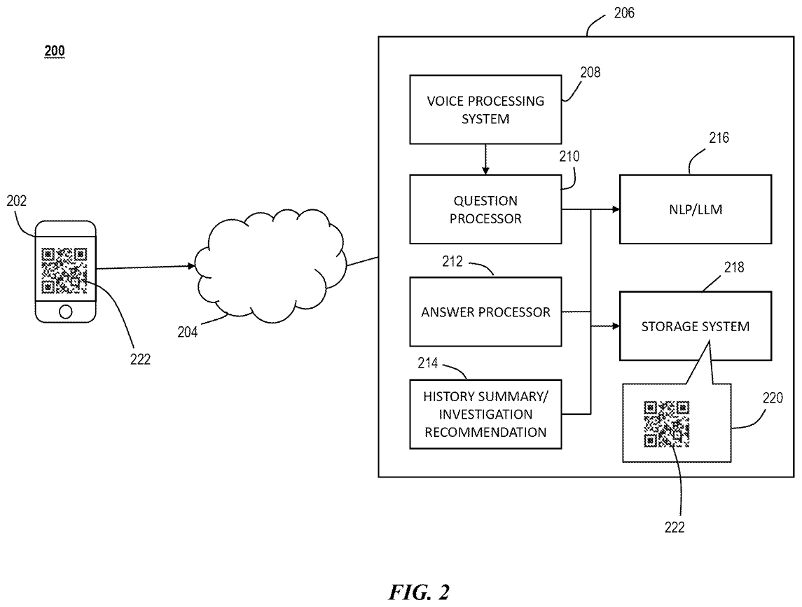

FIG. 2 shows a block diagram of an example of a medical record system 200, in accordance with some embodiments. In particular, the medical record system 200 automates medical history documentation by generating a record, and then automatically presenting relevant portions of the record to various personnel as needed in order to eliminate duplication of effort and opportunities for mistakes. There is a medical data center 206 that includes various computing resources for initiating or updating a patient record. As an example, a person, at home or otherwise remotely located from a medical facility associated with the data center 206, uses a computing device 202 to access the data center 206 over the Internet 204 or equivalent wide area network. The computing device 202 can be, for example, a cellular telephone (e.g., “smartphone”) device that has installed an application program designed to interact with the data center 206 resources. However, other types of computing devices can be equivalently used to interact with the data center 206. The interaction can be automated, allowing the user to speak into their computing device 202. For example, upon logging into the data center and providing identification information, the data center can issue a voice prompt, or an instruction to generate a voice prompt at the computing device 202, to describe what is wrong. In response, the user of the computing device can verbally describe their ailment, and their speech can be recorded, encoded, and transmitted to the data center for processing using a voice processing system 208, in conjunction with a natural language processor and large language model 216. Several instances of the natural language processor and large language model can be used to accommodate various spoken languages. Once having received the initial description, the voice processing system 208 can parse the received speech to identify key words. Based on the identified keywords, follow up questions can be generated by a question processor 210, and responses to those questions can be processed by an answer processor 212. The question generator 210 is designed along a question tree that branches depending on the responses to obtain relevant information for diagnoses. The answer processor 212 feeds back to the question processor 210 for follow up questions. For example, in the initial description of the ailment, the user could indicate abdominal pain, A follow up question would be, for example, “is the discomfort on a side, or more in the middle?” On the other hand, the user could indicate that they think they have a broken bone, which will start the question processor at a different diagnostic start point. Routine questions such as which medications the user is taking, allergies, surgical history, etc. can also be used to gather relevant information. During this process, the data center 206 is storing received information in a temporary file that can be duplicated at the computing device 202. The information management is handled by the history summary processor 214, which can also generate initial recommendations for investigation, and initial diagnoses. The process isn't intended to be comprehensive, just to obtain the initial information that would normally be asked at the initial intake at an emergency room in a hospital, for example. The process can generate an encoded identifier such as a QR code 222 that is used to access the file and which can encode some of the information. The identifier 222 is intended to be used by scanning equipment, and is a visual identifier. The identifier 222 is sent to the user's computing device 202 so that the user can present the identifier for scanning upon entry into a medical facility associated with the data center. The data center 206 can operate with several medical facilities and track the number of patients being treated at each facility, and based on that recommend a particular one of several medical facilities for the user to visit. A storage system 218 can include one or more databases in which the user's file 220, including the identifier 222, is stored and safeguarded in accordance with relevant privacy rules.

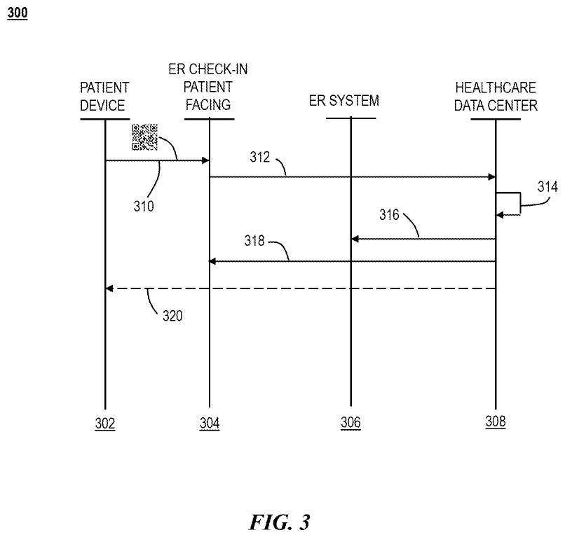

FIG. 3 shows a chart of an initial process 300 for receiving a patient at a medical facility, in accordance with some embodiments. The chart indicates several entities, including the patient device 302 (e.g. 202 of FIG. 2). There is also patient-facing check-in system 304, for use by arriving patients, which is located in an intake facility such as an emergency room of a hospital. The check-in system 302 includes an optical scanner to scan the patient's identifier 222 on their device. There is a provider facing system 306 at the entry location, for use by intake personnel and triage, and of course the data center 308 (e.g., 206 of FIG. 2). After performing the initial process outlined in relation to FIG. 2, the patient then has an identifier 222 on their mobile device 202. Upon arriving at the facility, the patient device 302 presents the identifier for scanning at 310 to the check-in equipment 304. The check-in equipment can decode the identifier, or transmit the identifier information to the data center 308 in 312. In response, the data center 308 can locate the patient's record, a history of present illness (HPI) in 314. Further, the data center can send information to the provider system 306 to let personnel know that the patient has arrived in 316, and an indication of the present ailment. The data center 308 also acknowledges this in 318 to the check-in equipment so that the patient knows they have been checked in, and will then wait to be seen or sent to a bed/room. In some embodiments the data center may also send a message or notification to the patient's device in 320 to confirm that they have been received. Afterwards, the patient may proceed to triage or wait to be seen.

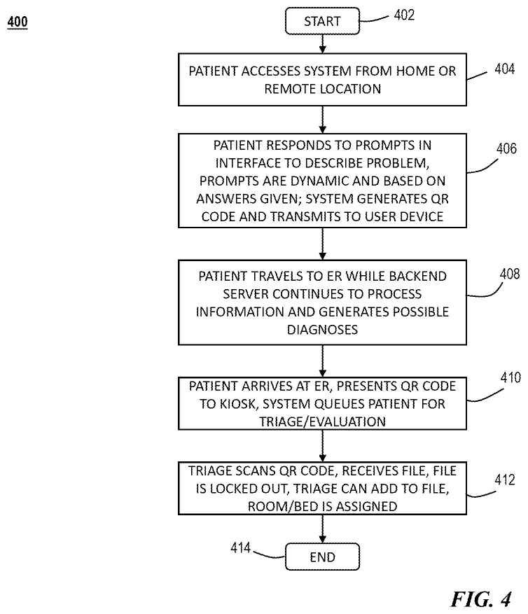

FIG. 4 shows a flow chart diagram of a method 400 for receiving a patient at a medical facility, in accordance with some embodiments. Method 400 encapsulates the processes discussed in relation to FIGS. 2 and 3. At the start 402, the patient has determined that they are experiencing an ailment and have decided to seek examination and treatment from a medical care provider. As the ailment can be something that has arisen or occurred very recently, the patient may need to be seen as soon as possible, rather than, for example, calling and making an appointment with their general care physician. At step 404 the patient uses their computing device to access the care provider data center, which can be facilitated by an application program designed to run on the patient's computing device. Thus, the patient's device is a client device that is remotely located from the data center. In response to accessing the data center, the data center, using voice or text prompting, elicits initial information from patient, including identity information, and information about the present illness/ailment. In addition to speech input, the data center may request that the user collect images of the affected area, if appropriate. In some cases, progression of a symptom, such as swelling or inflammation evidence at the patient's skin may be important. Thus, one or more photographs can be taken and transmitted to the data center for inclusion in the HPI record. In response, the patient receives an identifier such as a QR code that can be displayed on a graphical display of the patient's device. In step 408 the patient can travel to the appropriate medical care facility, and in that time the data center can continue to process the received information to identify possible diagnoses for investigation upon being seen by a care provider/physician at the medical care facility. In step 410, the patient has arrived at the medical care facility, and presents the identifier to a check-in system, such as a kiosk, for scanning. The scanned identifier is used to retrieve information from the HPI record and provides the information to the administrative and triage computer devices. Alternatively, upon being seen by the triage personnel, in step 412 the patient's identifier can be scanned to retrieve the patient's HPI record. After a brief review the triage personnel can confirm the symptoms, and indicate an updates/changes in symptoms or severity of symptoms. In the end 414 the patient assigned and taken to an examining station (e.g., bed/room).

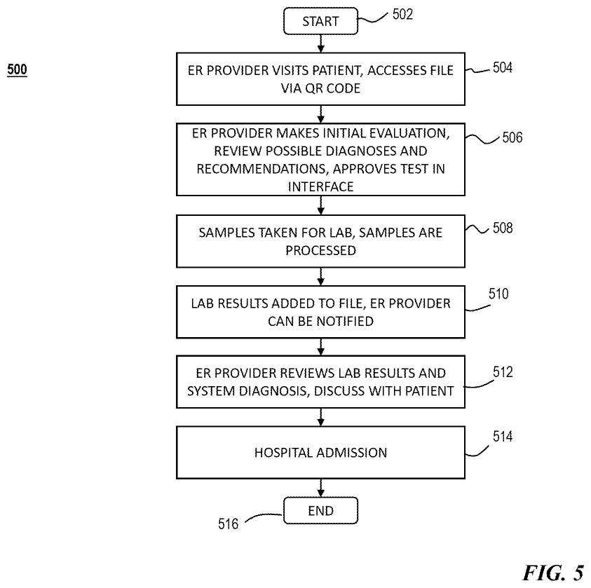

FIG. 5 shows a flow chart diagram of a method 500 for processing a patient in a medical facility, in accordance with some embodiments. The method 500 relates to the steps taken in the medical facility once the patient is assigned to an examining location at the medical facility. Thus, at the start 502, method 400 has been completed and the patient is location in an examining location. In step 504 a physician or other medical care provider visits the patient for an examination, and can access the patient's HPI file using the identifier, or the file can be pushed to the care provider's mobile computing device through an intranet that has wireless access points. In step 506 the care provider makes an initial examination, based on the HPI file information, review possible diagnoses recommended by the data center after processing the initial HPI information provided by the patient, and can rule out or select diagnoses for further investigation, or enter a new possible diagnosis. To confirm or rule out diagnoses, the care provider can then order one or more tests to be run, such as blood tests. The test order is provided to the data center through the care provider's computing device, and noted in the patient's HPI record. In addition, the data center can initialize test materials, such as by pushing the patient's test order information to the testing personnel, where, for example, labels can be printed for sample collection containers. In step 508 the required samples can be taken from the patient and processed by the laboratory to obtain results, and in step 510 the results can be automatically sent by the sample processing equipment to the data center, and the data center can push a notification to the care provider that the test results for the patient are available for review, which can be done at the care provider's device in step 512. The test results can be discussed with the patient and appropriate care can the be prescribed and further treatment provided. The testing can include imaging of bodily portions of the patient to determine the presence and/or extent of physical trauma or other abnormalities. In step 514 the patient can either be admitted to the hospital for further care and observation, or released to return home, and the method 500 ends 516.

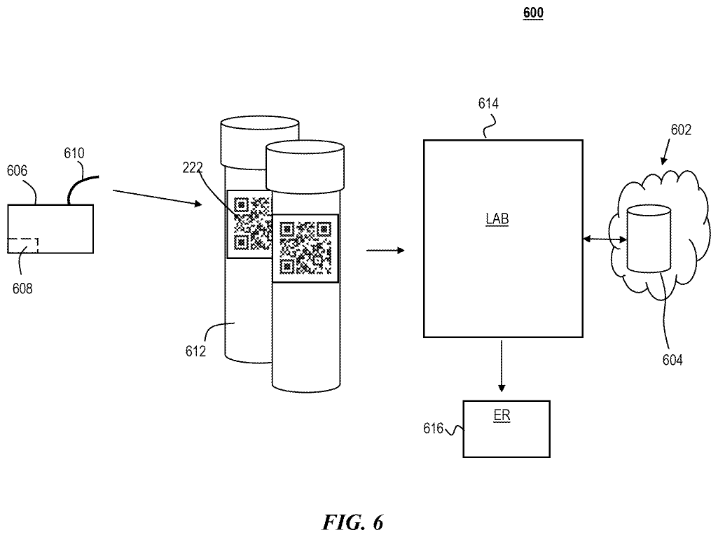

FIG. 6 shows a diagram illustrating a process 600 for initiating, tracking, and processing laboratory samples in relation to diagnosing a patient, in accordance with some embodiments. The data center 602 can be a cloud-based system including data stores 604 for retaining and providing patient records. In step 506, when a care provider orders one or more tests, the data center 602 can automatically transmit patient information to computing equipment at a test station. In response, the equipment can prepare test materials, such as containers for collecting samples from the patient. Thus, a printer 606 can be used at the test station to print labels 610 that include the patient identifier, as well as patient information in human language. The printer 606 can include a processor 608 for generating the label 610. The label, including the identifier 222, can then be applied to a sample container 612, and taken to the patient to collect the sample. In some cases, the patient is taken to a testing machine, which can include imaging machines such as X-ray and other imaging machines. The collected sample is then taken to a processing lab 614 where, in some cases, the testing of the sample can be automated. In the case of blood testing, for example, the container 612 including a blood sample can be inserted into a machine that automatically scans the identifier 222 and then provides test results to the data center 602 for inclusion in the record for the patient corresponding to the identifier 222. A notification can be pushed by the laboratory or testing facility to the care provider 616 or other emergency room personnel for review by the care provider attending to the patient. Thus, the process 600 applies generally to testing, whether it is vision testing, blood testing, imaging, and any other form of testing.

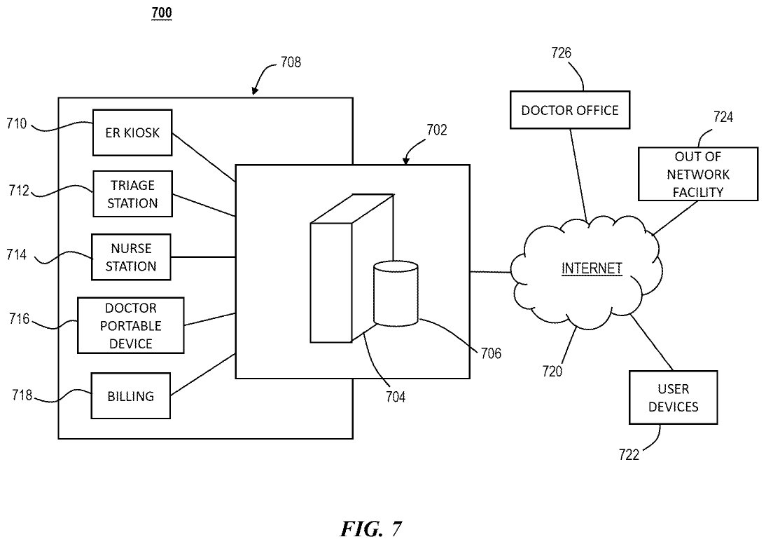

FIG. 7 shows a network diagram of a system 700 for initiating and using medical records, in accordance with some embodiments. They system 700 indicates a general network architecture in which the data center 702 can operate. The data center 702 can include one or more servers 704 and databases 706. The data center 702 is coupled to a wide area network 720 such as the Internet, so that remotely located user device 722 can access the data center. And further, the data center can be accessed by, or access other facilities 724, such as if a patient record needs to be transferred to a different facility/network. In addition, for example, physicians can access the data center from their remote offices, such as when the patient visits the office for follow up examination and/or treatment.

The data center 702 is coupled to an intranet or a virtual intranet (e.g., virtual private network over a wide area network) 708 that is operable in one or more medical facilities. Each facility can include both wired and wireless networking equipment for all of the various computing resources to access the data center 702. For example, the check-in kiosk 710, triage station 712, nurse station 714, physician's portable device 716, and the billing system 718 can all operate in the intranet 708 to access the data center. Real time monitoring devices 719, like EKG, blood pressure, blood oxygen saturation, among others can provide real time data to the data center that is recorded in the patient's record.

In the foregoing description it will be understood that robust security measures and compliance with global data protection regulations (e.g., GDPR, HIPAA) to protect patient data are used and enforced. This includes the use of encrypted databases and secure connections for data transmission. Thus, the communications described over both Internet and intranets are secured, and the database where the records are stored are suitably encrypted.

The system can be integrated not just with hospital admission systems but also with Emergency Room Management Systems (ERMS) and Hospital Information Systems (HIS) to automate bed assignments and healthcare provider assignments. This integration allows for dynamic allocation of resources, which can significantly reduce patient wait times and optimize hospital workflows. In other words, the data center can access information indication room/bed availability and assign the patient to a bed, such as when being admitted to the hospital in step 514. Resource databases in general can be accessed and reserved for the patient through the data center as part of an automated flow process that avoids the need for personnel to make decision and enter information. This greatly reduces the otherwise redundant operations commonly associated with hospital operation.

The system has capabilities for generating comprehensive assessment and management plans based on the synthesized medical history and investigation results. This would not only support initial diagnosis but also enable ongoing care management, integrating predictive analytics to foresee patient care needs. As information is added to the patient's record, the assessment can be refined using artificial intelligence systems. Thus, the system uses artificial intelligence for enhanced diagnostic support. The system employs advanced machine learning algorithms to predict potential urgent care needs based on symptom onset and progression, enabling pre-emptive care measures and more personalized patient care strategies.

The system can integrate with real-time monitoring devices, providing continuous data that feeds into the patient's history for ongoing assessment without manual input. This can be pivotal in acute care settings where condition changes rapidly. Thus, the system reduces administrative burdens by automating data capture from patient wearables and other health monitoring devices, which feeds directly into the patient's electronic health record (EHR), ensuring data is up-to-date and reducing redundancy.

A method and system for automating medical history documentation has been disclosed that allows a person to initiate care remotely by accessing a data center associated with the medical care enterprise and input various information describing their illness or ailment. The system can use voice processing to make the process easy and efficient. Once a person begins describing their symptoms, the data center can process the information to identify key words and generate follow up questions according to various diagnosis branches. The data center also generates an identifier to be used by the patient upon arriving at the medical care facility to avoid the typical wait times associated with conventional intake. The patient instead simply has their device display the identifier and it is scanned to initiate the triage and examination process. Thus, the triage personnel do not need to again ask the patient all the same questions, and likewise the examining physician will have all of the patient's background medical and medical history presented to the physician without the physician having to also ask questions of the patient. The system and method provide numerous benefits to the patient and the medical care enterprise.

The system addresses the inefficiencies highlighted in the problem landscape by revolutionizing the workflow of medical history taking and documentation. It leverages advanced technology to mimic the thoughtful and dynamic process of a physician, beginning right from Stage 0, where the patient's journey to seeking care starts.

Unlike traditional systems that rely on a predetermined set of questions, The system offers a dynamic approach to history taking. This means that the system doesn't just follow a rigid script but adapts the questions based on the patient's initial inputs and responses. This method is much closer to how a real consultation with a physician would unfold, where the doctor tailors their questions based on the conversation flow and the patient's specific symptoms.

As the patient inputs their chief complaints, The system intelligently generates follow-up questions that are relevant to the symptoms presented. This approach ensures that the history taken is comprehensive and tailored to the individual, rather than a one-size-fits-all questionnaire.

For returning or existing users, the system seamlessly integrates information from previous records. This means that patients are not asked to repeat information that the system already knows, reducing redundancy and enhancing the efficiency of the history-taking process.

By collecting registration details upfront, the system saves valuable time at the registration stage in the ER or clinic. This streamlined approach minimizes the administrative burden both on the patient and the healthcare providers, allowing more focus on immediate care needs.

The system's AI-driven system uses the information gathered to help rule in or out different possible diagnoses. This is achieved through targeted questions that delve deeper into the symptoms and their associations, guided by medical logic and diagnostic algorithms.

After the consultation, the system generates a comprehensive history summary, including HPI, past medical, social, family, and other details, which is encapsulated in a QR code. Healthcare providers can quickly scan this QR code to download and review the detailed history directly when opening the H&P document in the EMR, significantly reducing the time needed to access critical patient information. By automating the comprehensive gathering and documentation of patient history, the system not only streamlines the diagnostic process but also reduces the workload on healthcare providers. Each provider can save 15-30 minutes per patient, which accumulates significant time savings over multiple patient interactions. This reduction in redundant processes and time savings significantly reduces provider burnout, enhances patient care, and optimizes the overall efficiency of medical practices.

In traditional settings, when a patient speaks a different language, healthcare providers often rely on third-party translation services. This typically involves scheduling a video call with a translator who mediates the conversation between the patient and the physician. This process introduces significant delays between the patient's responses and the physician's questions, prolonging the consultation time and potentially leading to inaccuracies due to multiple steps in communication. Relying on external translation services can introduce unpredictability in terms of availability and quality. During peak hours or in less common languages, the wait times for a translator can significantly delay care. The system integrates real-time translation capabilities directly into its interface, supporting multiple languages in both speech and touch interactions. This allows patients to communicate their symptoms and medical history in their native language, which the system instantly translates into the physician's language. This feature eliminates the need for an intermediary translator, significantly reducing the time taken for consultations. By facilitating direct and immediate translation, the system enhances the accuracy of symptom reporting and medical history documentation. Patients can express themselves more naturally and comfortably, which improves the quality of the information gathered and increases patient satisfaction. The immediate availability of translated information streamlines the diagnostic process. Healthcare providers can access accurate and comprehensible patient data without delay, enabling faster decision-making and treatment initiation. This is particularly beneficial in urgent care settings where time is critical. The system's ability to handle multiple languages not only breaks down language barriers but also demonstrates cultural sensitivity, an important aspect of patient-centered care. This capability ensures that healthcare facilities can serve a more diverse patient population effectively. The real-time translation feature of the system can be customized to include additional languages as needed, making it a scalable solution that adapts to the changing demographics of the patient population.

By supporting both speech and touch inputs, the system accommodates different user preferences and accessibility needs, making the system user-friendly for a wide range of patients, including those who may have difficulties with traditional typing or touch interfaces.

With the ability to initiate lab tests and other diagnostic procedures during the patient's waiting period, results are obtained faster, accelerating the decision-making process. This rapid turnaround is crucial for conditions where timely intervention can significantly affect outcomes, such as in cases of acute appendicitis or myocardial infarction. The system's AI system can suggest specific investigations based on the differential diagnoses it generates. For instance, if the AI identifies symptoms correlating with appendicitis, it can recommend an abdominal ultrasound or a CT scan, which can be initiated immediately upon the patient's arrival.

The system significantly enhances the efficiency of emergency room operations by optimizing the protocols for early medical investigations. This optimized process impacts the entire admission flow, ensuring that patients receive faster and more effective medical attention. Here's how the system's features contribute to this improvement:

By providing comprehensive medical histories before the patient arrives at the ER, The system enables the medical team to prepare in advance for the patient's arrival. This pre-arrival information includes potential diagnoses and necessary medical background, allowing healthcare providers to plan and prioritize the required investigations without delay.

Hospitals can enhance operational efficiency by setting up dedicated areas equipped with the system kiosks and lab draw stations. These areas are specifically designed to expedite the process of conducting initial investigations as soon as the patient's history is reviewed. This setup reduces the logistical bottlenecks often encountered in traditional ER settings.

By enabling quicker diagnostic processes and decision-making, the system helps to reduce the time patients spend waiting for initial treatment and decisions regarding their admission or discharge. This efficiency not only improves patient satisfaction but also increases the turnover rate in the ER, allowing the facility to treat more patients effectively.

Early investigations mean that decisions about whether to admit a patient can be made swiftly, reducing unnecessary extended stays in the ER. This quicker throughput directly reduces congestion and improves the availability of ER resources for other patients.

With comprehensive histories and early diagnostic data available, physicians and consultants can concentrate on patient care rather than spending time gathering redundant information. This shift not only enhances the quality of care but also reduces the cognitive load on healthcare providers, potentially decreasing burnout.

By integrating the system into their protocols, hospitals can achieve a more streamlined, efficient, and patient-centered admission process. The early initiation of tailored investigations, guided by precise AI-driven recommendations, ensures that each patient's care pathway is optimized from the moment they enter the ER. This approach not only improves the effectiveness of medical interventions but also enhances overall hospital efficiency, making the system a transformative solution in emergency care settings.

The system leverages the power of synthetic data to revolutionize the accuracy and efficacy of medical diagnostics through its AI-driven platform. Here's an expanded look at how this works:

The system utilizes an advanced algorithm to generate synthetic datasets that mimic real patient scenarios. These datasets are crafted based on a diverse range of validated question-response narratives that cover various medical conditions and scenarios. This method provides a controlled environment to simulate a wide array of medical situations, from common conditions to rare diseases.

The synthetic datasets are meticulously designed to encompass a broad spectrum of symptoms, patient demographics, and clinical outcomes, ensuring that the AI model is trained on as diverse and comprehensive a dataset as possible. This prepares the system to handle real-world scenarios with greater accuracy.

y continuously incorporating new synthetic data, the system ensures that its AI models are not only robust but also adaptable to new medical insights and evolving healthcare practices. This ongoing process of fine-tuning enhances the AI's diagnostic algorithms, making them more precise over time.

The system integrates feedback from actual clinical use back into the system. This real-world data helps to further refine the synthetic models, ensuring that the AI's questioning logic and diagnostic predictions remain aligned with current medical standards and practices.

Each synthetic dataset and the resulting diagnostic algorithms are rigorously validated by a team of experienced physicians. This validation ensures that every aspect of the system's diagnostic process adheres to the highest standards of medical accuracy and is clinically relevant.

The physician review process also ensures that the questions generated by the system are practical and intelligible in a real-world clinical environment, facilitating ease of use and understanding among healthcare providers.

Unlike many diagnostic systems that rely solely on symptom-to-condition mapping, The system provides a layered reasoning behind each diagnostic suggestion. This includes detailing why certain conditions are considered over others based on the patient's specific symptoms and history, akin to a physician's thought process.

By transparently showing the reasoning behind its diagnoses, The system helps build trust among healthcare providers. This transparency is crucial for clinical acceptance and for providers to feel confident in relying on AI-assisted diagnostics.

The system's diagnostic reasoning and the resulting suggestions are designed to be seamlessly integrated into existing clinical workflows. This means that the system's outputs can be directly used by physicians to inform their decision-making process without disruption.

The integration of detailed diagnostic reasoning into the clinical workflows allows healthcare providers to make quicker, more informed decisions. This efficiency is crucial in emergency settings where time is of the essence and can significantly impact patient outcomes.

The system utilizes a 3D avatar to simulate conversations with patients, making the digital interaction more relatable and less mechanical. This can reduce the anxiety associated with medical data entry and increase user engagement, especially for patients uncomfortable with traditional digital interfaces.

Highlight the capability of the system to offer customizable avatars, which can be tailored to reflect the demographics or preferences of the patient population, further enhancing the personalized experience.

The system simplifies the entry of medical history by allowing patients to directly input symptoms via an intuitive mobile app or kiosk interface. This direct input method can be contrasted with systems that require navigating through multiple screens or complicated medical jargon.

Highlight how the inputs are immediately processed to generate the History of Present Illness (HPI), minimizing the delay between data entry and document generation. This efficiency is crucial in emergency settings where time is of the essence

The system directly integrates the generated History of Present Illness (HPI) into the hospital's admission system. This significantly reduces the redundancy typically encountered during patient admissions, where patients often have to repeat their symptoms and medical history multiple times to different healthcare providers.

By automating the initial patient evaluation and triage based on the comprehensive HPI generated, the system reduces the workload on emergency room staff and speeds up the decision-making process for patient care prioritization.

The claims appended hereto are meant to cover all modifications and changes within the scope and spirit of the present invention.

What is claimed is:

1. A method for automating medical documentation, comprising:
receiving, at a data center, speech provided by a patient computing device located remotely from the data center via a wide area network;
processing said speech to identify keywords relating to at least one medical condition;
generating at least one follow up question in a natural language prompt that is transmitted to the patient computing device and receiving follow up information via speech from the patient computing device;
generating a visual identifier that is then associated with a record created to include the relevant information obtained from the speech and transmitting the visual identifier to the patient computing device;
receiving, at the data center, from a check-in kiosk at a medical facility associated with the data center, information from the visual identifier; and
the data center, in response to receiving the information from the visual identifier, accessing the record and pushing information from the record to at least one computing device associated with medical care provider at the medical facility, wherein pushing the information prevents the need to ask a patient associated with the patient computing device questions that have been answered.

2. The method of claim 1, further comprising:
the information being accessed at the at least one computing device associated with the medical care facility,
receiving input at the at least one computing device associated with the medical care facility for at least one test, wherein the input is transmitted to the data center as a test order, and
in response to receiving the test order, the data center automatically transmitting information to a computing device for generating testing materials.

3. The method of claim 1, wherein the speech is provided in a language selected by a patient at the patient computing device.

4. The method of claim 1, wherein the patient computing device is a mobile phone device.
